(19) United States
(12) Patent Application Publication
(10) Pub. No.: US 2002/0095107 A1
(43) Pub. Date: Jul. 18, 2002

(54) Viscoelastic lined elastic sleeve for the protection or treatment of fingers or toes

(76) Inventor: Jean-Luc Martin, Loriol (FR)

Correspondence Address: James E. Nilles, Nilles & Nilles, S.C., Firstar Center, Suite 2000, 777 East Wisconsin Avenue, Milwaukee, WI, 53202, US

(21) Appl. No.: 10/035,384

(22) Filed: Nov. 9, 2001

Related U.S. Application Data
(63) Continuation of application No. PCT/FR00/01359, filed on May 19, 2000.

(30) Foreign Application Priority Data
May 21, 1999 (FR) 99/06828

(51) Int. Cl. A61F 13/00; A61F 13/06

(52) U.S. Cl. 602/61

(57) ABSTRACT

A sleeve for the protection or treatment of fingers, toes or other parts of the body. The sleeve has at least one piece of fabric containing thermoplastic fibers, assembled by welding along its edges to form all or part of the body of the sleeve, and at least one layer of viscoelastic gel arranged on one side of the piece of fabric.

### CROSS-REFERENCE TO A CO-PENDING APPLICATION

This application is a continuation of international application No. PCT/FR00/01359, filed on May 19, 2000.

### BACKGROUND OF THE INVENTION

1. Field of the Invention

The present invention relates to the field of dressings in the form of sleeves for the protection and treatment of fingers, toes and, more generally, members of the human body.

The extremities of the members of the human body are exposed to stresses and strains leading to injuries or complaints that require protection or treatment.

2. Description of the Related Art

For this purpose, there are various structures and shapes of sleeves containing viscoelastic materials. In particular, there are tubular sleeves constituted solely of viscoelastic polymers. The inconvenience of these sleeves is that the polymer tube must have the mechanical properties of elasticity, resistance, viscoelasticity and compactness that are contradictory and are not combined satisfactorily to the detriment of the physiological effects of the sleeve. There are also woven sleeves containing a viscoelastic material lining. These sleeves are inconvenient in that they are not compact, as the fabric and the lining must be of substantial thickness due to the poor mechanical properties of the materials used. Furthermore, to solve the problem of the industrial production of a tubular sleeve made of fabric, large knit fabrics are used to the detriment of the thickness and the resistance of the sleeve and of the comfort of the user.

### OBJECTS AND SUMMARY OF THE INVENTION

Therefore, one object of the present invention is to produce a sleeve for the protection or treatment of fingers or toes that optimises the parameters of protection and comfort.

More particularly, one object of the present invention is to provide a protective sleeve that is not very thick while providing high resistance, great flexibility, good hold on a finger or toe, good adaptability to the various sizes of fingers or toes and excellent compatibility with skin including in the event of a lesion.

One main object of the present invention is to reduce the size and the inconvenience of a sleeve without affecting the effectiveness.

One particular object of the present invention is to provide a sleeve that offers protection replacing that of the epidermis, i.e. capable of absorbing friction and spreading out mechanical stresses.

Another object of the present invention is to produce a sleeve that is washable and reusable without losing its qualities.

Yet another object of the present invention is to provide a collective manufacturing method of sleeves at low cost price.

To achieve these objects, one idea of the present invention is to produce all or part of the body of the abovemenioned type of sleeve by means of a fabric containing thermoplastic fibres, offering advantageous characteristics of heat weldability (heat welding), slight thickness and mechanical resistance while being washable. Preferably, such fabric also includes elastic fibres in order to obtain the elasticity required to keep the sleeve on a finger or toe. Moreover, another idea of the present invention is to coat all or part of the inner or outer surface of such a sleeve with a viscoelastic gel, preferably a silicone gel with mechanical properties that are similar to the human plantar cushion, to provide ideal protection from epidermic stress.

More particularly, the present invention provides a sleeve for the protection or treatment of fingers, toes or other parts of the body, comprising at least one piece of fabric containing thermoplastic fibres, assembled by welding along its edges to form all or part of the body of the sleeve, and at least one layer of viscoelastic gel arranged on one side of the piece of fabric.

According to one embodiment, the sleeve comprises two pieces of fabric welded together along two edges, with one of the pieces of fabric bearing the layer of viscoelastic gel, and the welding areas of both pieces of fabric being set back from the edge of the layer of gel.

According to one embodiment, at least one of the two pieces of fabric contains elastic fibres.

According to one embodiment, the sleeve comprises one piece of folded fabric containing elastic fibres and thermoplastic fibres, having two opposite, welded edges, and bearing the layer of viscoelastic gel on one of its sides.

According to one embodiment, the viscoelastic gel is a silicone gel.

According to one embodiment, the silicone gel has a modulus in compression and a torsional modulus ranging from half to double the corresponding modulus of the human plantar cushion.

According to one embodiment, the viscoelastic gel is a self-adhesive silicone gel.

According to one embodiment, the layer of viscoelastic gel is covered with a removable protective film.

According to one embodiment, the sleeve comprises at least one piece of fabric containing a mixture of polyamide fibres and elastane fibres.

According to one embodiment, the sleeve is reversible and suitable for use on the right side with the layer of viscoelastic gel facing inwards and suitable for use on the wrong side with the layer of viscoelastic gel facing outwards.

According to one embodiment, the sleeve comprises two superimposed layers of viscoelastic gel on at least one area of its inner or outer side.

According to one embodiment, the sleeve comprises two layers of viscoelastic gel combined by inclusion, one being of a smaller surface area and included in the other.

The present invention also relates to a manufacturing method of a sleeve for the protection or treatment of fingers, toes or other parts of the body, including steps of welding together two panels of fabric containing thermoplastic fibres by applying heated tips to the surface of a first panel of fabric at regular intervals corresponding alternately to a semi-circumference of the sleeve and to a cut-out distance of sleeves, and fixing at least one layer of viscoelastic gel onto the free surface of the second panel of fabric, by covering the spaces located at right angles to the welding areas.

According to one embodiment, the method includes a step of cutting out the compound formed by the assembled panels of fabric by following cutting lines located between two weld lines, to obtain portions of compound in the form of flattened tubes that can then be cut into individual sleeves of the required length.

According to one embodiment, the width of the heated tips ranges from a few tenths of a millimeter to a few millimeters.

According to one embodiment, the width of the intervals corresponding to the cut-out distances of sleeves is in the order of a few millimeters.

The present invention also relates to a manufacturing method of a sleeve for the protection or treatment of fingers, toes or other parts of the body, including steps of arranging at least one layer of viscoelastic gel on one side of a panel of fabric containing thermoplastic fibres, without covering two opposite edges of the panel of fabric with gel, folding the panel of fabric so that each edge is divided into two parts located opposite each other, and welding the respective parts of each edge that are opposite each other together.

According to one embodiment, the welding areas of the parts opposite each other are very near the edges of the layer of gel.

### BRIEF DESCRIPTION OF THE DRAWINGS

These and other objects, advantages and features of the present invention shall be presented in greater detail in the following description of two embodiments of sleeves according to the present invention and of manufacturing methods of these sleeves, provided in relation with, but not limited to, the following figures:

FIG. 1 is a view in perspective of a first type of sleeve according to the present invention, represented flat before being turned out,

FIG. 2 is a cross-section view of the sleeve in FIG. 1 after being turned out,

FIGS. 3a and 3b show two uses of the sleeve in FIG. 1,

FIGS. 4 and 5 show a manufacturing method of the sleeve in FIG. 1,

FIGS. 6 and 7 show a manufacturing method of a second type of sleeve according to the present invention,

FIG. 8 is a view in perspective of the second type of sleeve according to the present invention, and

FIGS. 9 and 10 represent two variations of embodiments of sleeves according to the present invention, applicable to the sleeves of the first or second type.

### DESCRIPTION OF THE PREFERRED EMBODIMENTS

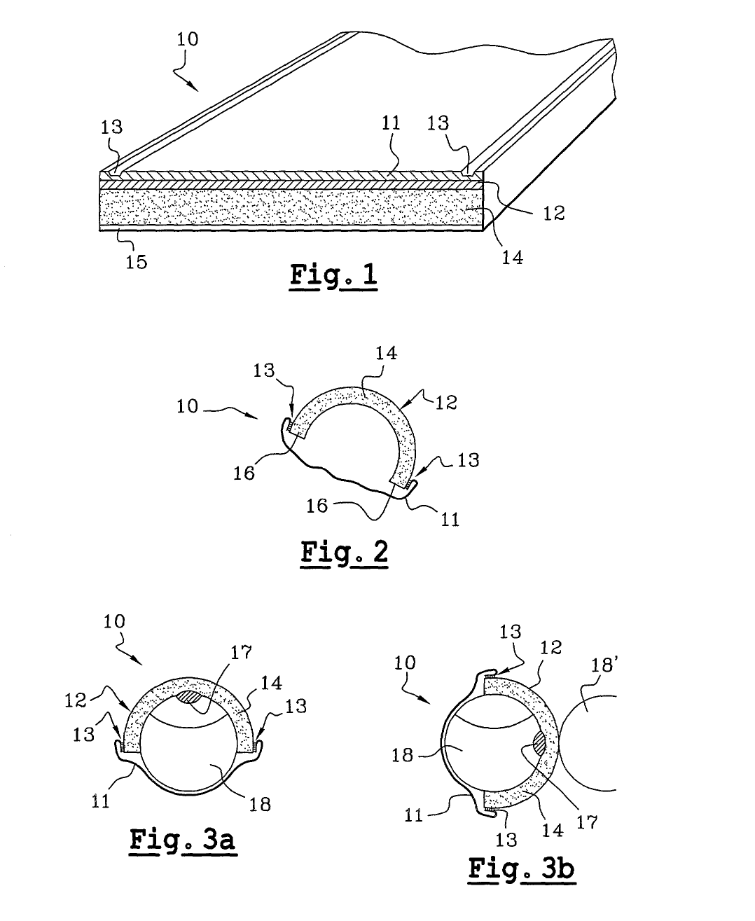

FIG. 1 is a view in perspective of a first type of sleeve 10 according to the present invention, represented flat before being turned out. According to a first aspect of the present invention, the body of the sleeve 10 is constituted here by two pieces of fabric 11, 12 heat welded together, the pieces of fabric 11, 12 containing thermoplastic fibres for that purpose. Furthermore, at least one of the pieces of fabric, preferably both, also contains elastic fibres giving the sleeve 10 the elasticity required. Therefore, preferably, each piece of fabric contains a mixture of elastic fibres and thermoplastic fibres. The elastic fibres are preferably constituted of elastane and the thermoplastic fibres are constituted of polyamide. For example, the pieces of fabric each contain a mixture of polyamide and elastane fibres in respective proportions of 70% and 30%. Advantageously, the fabric of each piece 11, 12 is fine and its basis weight is preferably less than 250 g/m².

According to the embodiment shown in FIG. 1, both pieces of fabric 11, 12 are rectangular in shape and are carefully superimposed. The two pieces of fabric are assembled by two weld lines 13 carried out on the free side of the piece of fabric 11, each along a large side of the double piece of fabric. As explained below in greater detail, the presence of thermoplastic fibres in each of the pieces of fabric, particularly polyamide fibres, allows these welding areas to be achieved easily by simple localised heating leading to a fusion of the fibres.

According to another aspect of the present invention, one of the two pieces of fabric, here the piece 12, has, on its free side that is opposite the weld lines 13, a lining 14 constituted of a viscoelastic silicone gel. This lining 14 is, advantageously, in the form of an even layer of gel of a thickness ranging from a few tenths of a millimeter to a few millimeters, stuck onto the free side of the piece of fabric 12. The layer of silicone gel 14 preferably covers the whole surface area of the piece of fabric 12 and particularly covers spaces at right angles to the weld lines 13.

Here, the present invention is based on the fact that certain silicone gels have remarkable viscoelastic properties, similar to the mechanical properties of certain human cutaneous tissues. Among these viscoelastic silicone gels, the compositions of silicone polymer mixes described in patent number FR-2 712 487, to which reference shall be made for details concerning formulation and development, have the surprising particularity of reproducing the mechanical properties of the natural plantar cushion and in particular having similar values of modulus in compression and torsional modulus. Silicone gels with such formulations are marketed by the applicant under the name of EPITHELIUM 26®. Other formulations make it possible to obtain, sometimes to the detriment of the similarity of the mechanical modulus in compression and torsional modulus, intrinsic adhesion properties of the silicone gel. Such silicone gels are marketed by the applicant under the name EPITHELIUM 27 and EPIHELIUM 27+. They have exceptional contact qualities and are outstanding load spreaders capable of achieving remarkable results in terms of comfort and prevention and in the treatment of certain complaints, particularly hyperkeratosis. In practice, the conformity of these gels can be verified by checking the modulus in compression and the torsional modulus the nominal values of which are in the order of 4.10³ N/m² and 4.10³ Nim² to 15.10³ N/m², respectively. A tolerance of 50% in relation to threshold values of these moduli is accepted without losing the advantages of these gels for the medical or paramedical applications considered here.

Therefore, according to one preferred embodiment of the present invention, the gel forming the lining 14 of the sleeve 10 is a viscoelastic silicone gel the mechanical properties of which are in the order of those of the human plantar cushion, as the gel has, in particular, a modulus in compression and a torsional modulus ranging from half to double the corresponding modulus of the plantar cushion. For example, the gel is the abovementioned silicone gel marketed under the name EPITHELIUM 26.

In one variation of embodiment, the silicone gel has, in addition to mechanical properties similar to the human plantar cushion, the property of being intrinsically adhesive by the simple effect of being in contact with the skin, and the gel can be unstuck and re-stuck almost indefinitely. For example, the gel is the abovementioned silicone gel marketed under the name EPITHELIUM 27 OR EPITHELIUM 27+.

As explained below, two types of silicone gel can also be combined in a sleeve according to the present invention.

Preferably, a removable protective film 15 is applied to the free surface of the layer of silicone gel 14 to keep the surface clean. The film 15 is, for example, made of plastic particularly polyethylene.

On FIG. 1, the sleeve 10 according to the present invention is therefore flat, and the two pieces of fabric 11 and 12 are arranged one against the other with the layer of silicone gel 14 facing outwards. The sleeve is therefore assembled inside out with the weld lines 13 visible. It is possible to use the sleeve as it is by inserting a finger or toe between the two pieces of fabric 11 and 12, and as the layer of gel 14 is then facing outwards it comes into contact and protects a neighbouring finger or toe.

The sleeve 10 can also be turned out after removing the protective film 15. It is then arched in a rather tubular form as shown by the cross-section view in FIG. 2. More particularly, the sleeve 10 obtained has one portion of fabric 12 that is roughly semi-tubular bearing the silicone gel 14 on its inside, and one portion of fabric 11 forming the other half of the sleeve, that is slack in appearance while the sleeve has not been put onto a finger or a toe. As the sleeve is turned out, the layer of silicone gel 14 is then facing inside the sleeve.

Given that according to the preferred embodiment of the invention the layer of gel 14 covers the whole inner surface of the piece 12, the layer of gel 14 therefore covers, seen from inside the sleeve, the welding areas 13 of the pieces of fabric 11, 12, that are arranged outwards and set back from the edges 16 of the layer of gel 14.

On FIG. 3a, the sleeve 10 is represented folded out and placed around a finger or a toe 18 that has an injury 17, for example a dorsal corn. The pieces of fabric 11, 12 cooperate for the elastic hold of the sleeve around the toe 18. Here, the deformation of the sleeve 10 and the elastic holding tension are mainly born by the piece of fabric 11 which is free from gel, the other piece of fabric 12 mainly serving to support and keep the gel 14 against the part of the toe 18 that has the injury 17. The fabric of the piece 11 is therefore preferentially elastic while the piece 12 may also be so, but with a lesser necessity to be so.

Once arranged around the toe 18, the layer of viscoelastic silicone gel 14 then covers approximately half the inner surface of the sleeve 10 and the two pieces of fabric 11 and 12 form two semi-cylinders according to the most simple embodiment in which the pieces of fabric 11, 12 have the same dimensions, although different variations are possible. As it has already been stated, the welding areas 13 are arranged outwards and set back from the edges of the layer of gel 14, so that the latter is placed between the toe 18 and the buildup of thickness due to the assembly and the turning out of the pieces of fabric 11, 12.

Still as an example, FIG. 3b represents a sleeve placed around a finger or a toe 18 that has a corn between digits 17 that is opposite a neighbouring toe 18′. In this case, the sleeve is oriented so that the welding areas 13 of the pieces of fabric 11, 12 are not located in the space between the digits.

Generally speaking, it can be seen from the above that a sleeve according to the present invention is an advantageous response to the objectives set in the preamble. The sleeve is cortpact, very comfortable for fingers or toes, as the pieces of fabric are particularly fine and the layer of protective silicone gel is of a limited thickness, preferably in the order of a millimeter. This is due to the excellent mechanical characteristics of viscoelastic silicone gels, such as the gels EPITHELIUM 26, EPITHELIUM 27 and EPITHELIUM 27+ mentioned above as a non-restrictive example. The sleeve also holds extremely well in place, as the piece of fabric that is free from gel keeps all its elasticity. Furthermore, such a reserve of elasticity allows the number of sizes of sleeves to be reduced and thus to cover all sizes of fingers and toes with only three standard sizes. Also, the welding areas and turning out areas of the pieces of fabric of the sleeve do not cause any discomfort or undesired effect on the area of the finger or toe to be protected, as the viscoelastic silicone gel is inserted between them. Finally, the sleeve is preferably constituted of materials that make it anallergic, non-irritant and noncytotoxic. Moreover, these materials advantageously make the sleeve both washable and reusable.

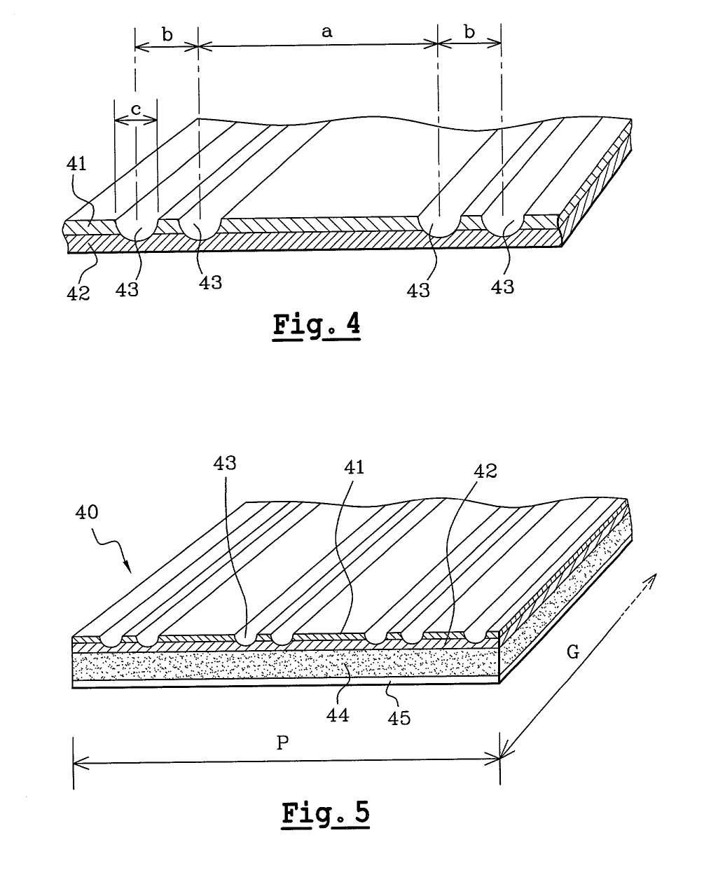

FIGS. 4 and 5 show a collective manufacturing method of the sleeve that has just been described. As shown in FIG. 4, the initial step of the method according to the present invention includes assembling two panels of fabric 41, 42 each composed, preferably, of elastic fibres and of thermoplastic fibres. As shown in FIG. 5, the width P of the panels of fabric 41, 42 is sufficient for several lines of sleeves of identical or different widths to be produced in parallel. The length G of the panels of fabric is defined by the needs of production that can, moreover, be carried out non-stop.

FIG. 4 shows the assembly of the panels of fabric 41, 42 in detail. The two panels are arranged flat, one on top of the other, and form a collective production panel of fabric 40 of a considerable width on which electrodes or heated tips similar to those of soldering irons are applied. The heating and indentation of each tip causes a localised fusion of the thermoplastic fibres of the two panels of fabric and forms weld grooves 43.

The heated tips may be in lines or pinpoints, and, in this case, the weld lines 43 are carried out by a relative linear displacement of the tips and of the surface of the fabric. They are positioned at regular intervals a, b corresponding respectively to the width or semi-circumference of each sleeve and to a distance between two sleeves on the collective production panel 40. Thus, weld lines 43 that are parallel and in pairs are achieved. The distance b between two grooves 43 is of a few millimeters, typically 2 mm to 5 mm. Preferably, the grooves 43 formed by the shape of the heated tips are of a width c in the order of a millimeter or a few millimeters, typically in the order of 0.5 mm to 3 mm. It is preferable for the sum of the distance b between two weld lines and of the width c of one weld line 43 not to exceed 6 mm as this dimension determines the excess fabric that the layer of gel must isolate from the finger or toe to be protected. Such an assembly step provides a set of flattened, attached tubes of fabric. The weld-free surface of the fabric 42, located under the fabric 41 in relation to the direction in which the tips are applied, is perfectly flat.

The following step of the method according to the present invention, shown in FIG. 5, includes sticking a layer of viscoelastic silicone gel 44 against the flat intact surface of the panel of fabric 42. Preferably, the free side of the layer of silicone gel 44 is protected by a removable plastic film 45, such as a polyethylene film, before sticking. At the end of the sticking step, a multilayer assembly of fabric/fabric/gel/film is obtained comprising a line of flattened sleeves that are inside out and not detached. The multilayer assembly can then be cut out along cutting lines located in the spaces b between the pairs of weld lines 43, to detach strips of flattened sleeves. Then, these strips can be cut to form sleeves of the required length.

This manufacturing method of sleeves according to the present invention is advantageous in that it includes simple steps for producing flat sleeves and allows the sleeves to be produced collectively at a low cost price.

However, other variations and embodiments of the present invention can also be considered.

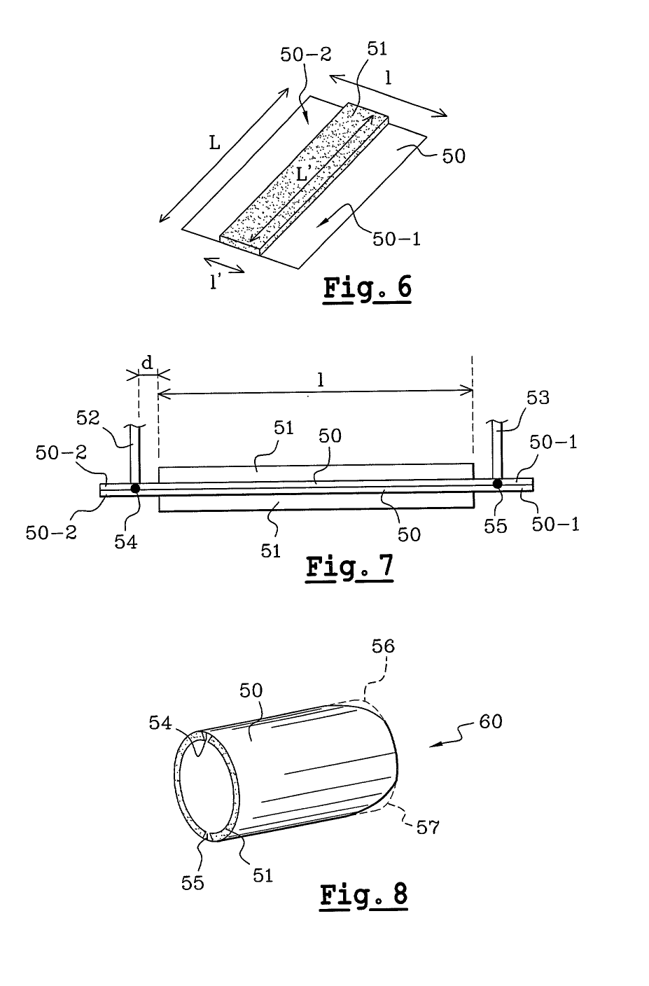

Thus, FIGS. 6 and 7 represent a manufacturing method of a second type of sleeve, with a closed end, produced by folding a piece of fabric.

As shown in FIG. 6, a rectangular piece of fabric 50 of a length L and a width l containing elastic and thermoplastic fibres is set out flat on a support. The piece 50 receives, on its free side, a rectangular strip of viscoelastic silicone gel 51 assembled by sticking. The strip of gel 51 is of a length L′ preferably equal to L and a width l′ that is less than l, and is centred on the piece of fabric 50 to leave two edges 50-1, 50-2 of the piece 50 visible, that are not covered with the gel. The strip of gel 51 is preferably covered by a removable protective film (not represented).

In the step shown in FIG. 7, the piece of fabric 50 is folded in two equal parts widthways l so that the halves of the edges 50-1, 50-2 are respectively opposite each other. Preferably, the folding is done in such a way that the strip of gel 51 is located on the outside of the sleeve being formed. The two halves of the edges of fabric 50-1, 50-2 are then pinched and welded one against the other by means of heated tips 52, 53 to form weld lines 54, 55 that can be seen in the cross-section view in FIG. 7. The tips 52, 53 are narrow, for example a few tenths of a millimeter, to obtain narrow weld lines 54, 55. As above, the weld lines 54, 55 can be continuous or pin points obtained by relative displacement of the tips 52, 53 and of the piece of fabric 50. Tips of a length in the weld plane corresponding to the area of the weld lines 54, 55 may also be used. The distance d between the weld strips 54, 55 and the edges of the strip of gel 51 is preferably very short, for example in the order of 0.5 mm. The parts of the edges of fabric 50-1, 50-2, which extend beyond the weld lines 54, 55 are then cut. The two corners of fabric located at the end of the weld lines 54, 55 at the closed end of the sleeve can also be cut, to obtain, at the closed end of the sleeve, bevelled corners 56, 57 shown in FIG. 8.

The characteristics, particularly the type and thicknesses of the materials used in this embodiment are chosen in accordance with the method set out above.

FIG. 8 shows the sleeve 60 obtained using this method, once it has been turned out. The sleeve 60 differs from the sleeve 10 described above by the fact that the layer of gel 51 covers almost all the inner surface except for the joining areas of edges 50-1, 50-2 of the piece of fabric 50, where the weld lines 54, 55 are located. However, the thickness of the strip of gel 51 prevents the bump formed by the joins from rubbing against the skin.

The method that has just been described lends itself, as the previous one, to a collective production of sleeves. Moreover, the strip of fabric 50 and the strip of gel 51 shown in FIG. 6 can be long and be cut to the required length after the welding step shown in FIG. 7.

It will be understood that variations of these embodiments of the present invention may be made. For example, two strips of fabric similar to the one shown in FIG. 6, each covered with a strip of gel, can be assembled one against the other and welded at their ends to obtain a sleeve comprising two semi-cylinders each with a layer of gel. Also, the piece of fabric 50 described above can be folded lengthways and have its opposite edges 50-1, 50-2 welded together to form a tubular sleeve, that can then be cut into several tubular sleeves that are shorter in length.

Moreover, the layer of silicone gel covering all or part of the inner or outer side of a sleeve according to the present invention (according to the direction in which the sleeve is used) may contain a combination of several strips of silicone gel, of the same type or otherwise.

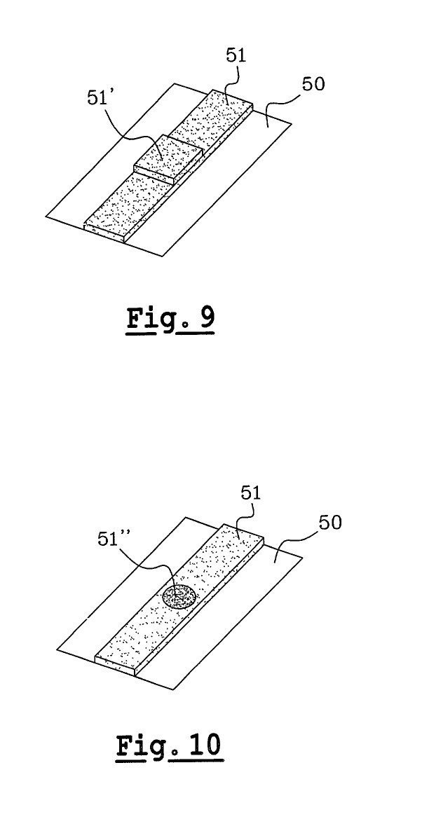

For a better understanding, FIG. 9 shows a strip of fabric 50 that is identical to the one in FIG. 6, bearing a strip of gel 51 that is covered locally, in its central part, by a second layer of gel 51′ of a shorter length, assembled by sticking. The additional layer of gel 51′ is, for example, square in shape and of a thickness ranging from 0.5 mm to 1.5 nm.

The strip of gel 51 and the additional layer 51′ can be of the same formlation, such as EPITHELIUM 26 for example. In this case, the layer 51′ is simply a reinforcement that provides greater protection to the end of the toe or finger against external strain. In particular, the layer 51′ is particularly useful against the micro-traumatisms of walking that cause blue nails.

The strip of gel 51 can also be of EPITHELIUM 26 and the additional layer 51′ of EPITHELIUM 27, or vice versa. In this case, the self-adhesive properties of EPITHELIUM 27 are combined with the load spreading qualities of EPITHELIUM 26 to totally prevent any rubbing on the surface of the skin.

Finally, another variation of embodiment shown in FIG. 10 consists in including, in the central part of the strip of silicone 51 arranged on the piece of fabric 50, a piece of silicone 51″ of a different type, for example including EPITHELIUM 26 in a strip of EPITHELIUM 27 or vice versa.

1. Sleeve for the protection or treatment of fingers, toes or other parts of the body, characterised in that it comprises at least one piece of fabric containing thermoplastic fibres, assembled by welding along its edges to form all or part of the body of the sleeve, and at least one layer of viscoelastic gel 14, 50, 51′, 51″) arranged on one side of the piece of fabric.

2. Sleeve according to claim 1, characterised in that it comprises two pieces of fabric assembled by welding along two edges, with one of the pieces of fabric bearing the layer of viscoelastic gel, and the welding areas of both pieces of fabric being set back from the edge of the layer of gel.

3. Sleeve according to claim 2, characterised in that at least one of the two pieces of fabric contains elastic fibres.

4. Sleeve according to claim 1, characterised in that it comprises one piece of folded fabric containing elastic fibres and thermoplastic fibres, having two opposite, welded edges, and bearing the layer of viscoelastic gel on one of its sides.

5. Sleeve according to claim 1, characterised in that the viscoelastic gel is a silicone gel.

6. Sleeve according to claim 5, characterised in that the silicone gel has a modulus in compression and a torsional modulus ranging from half to double the corresponding modulus of the human plantar cushion.

7. Sleeve according to claim 1, characterised in that the viscoelastic gel is a self-adhesive silicone gel.

8. Sleeve according to claim 1, characterised in that the layer of viscoelastic gel is covered with a removable protective film.

9. Sleeve according to claim 1, characterised in that it comprises at least one piece of fabric containing a mixture of polyamide fibres and elastane fibres.

10. Sleeve according to claim 1, characterised in that it is reversible and suitable for use on the right side with the layer of viscoelastic gel facing inwards and suitable for use on the wrong side with the layer of viscoelastic gel facing outwards.

11. Sleeve according to claim 1, characterised in that it comprises two superimposed layers of viscoelastic gel on at least one area of its inner or outer side.

12. Sleeve according to claim 1, characterised in that it comprises two layers of viscoelastic gel combined by inclusion, one being of a smaller surface area and included in the other.

13. Manufacturing method of a sleeve for the protection or treatment of fingers, toes or other parts of the body, characterised in that it includes the following steps of:
welding together two panels of fabric containing thermoplastic fibres by applying heated tips to the surface of a first panel of fabric at regular intervals corresponding alternately to a semi-circumference of the sleeve and to a cutout distance of sleeves, and
fixing at least one layer of viscoelastic gel onto the free surface of the second panel of fabric, by covering the spaces located at right angles to the welding areas.

14. Method according to claim 13, characterised in that it includes a next step of cutting out the compound formed by the assembled panels of fabric by following cutting lines located between two weld lines, to obtain portions of compound in the form of flattened tubes that can then be cut into individual sleeves of the required length.

15. Method according to claim 13 characterised in that the width of the heated tips ranges from a few tenths of a millimeter to a few millimeters.

16. Method according to claim 13 characterised in that the width of the intervals corresponding to the cut-out distances of sleeves is in the order of a few millimeters.

17. Manufacturing method of a sleeve for the protection or treatment of fingers, toes or other parts of the body, characterised in that it includes steps of:
arranging at least one layer of viscoelastic gel on one side of a panel of fabric containing thermoplastic fibres, without covering two opposite edges of the panel of fabric with gel,
folding the panel of fabric so that each edge is divided into two parts located opposite each other, and welding the respective parts of each edge that are opposite each other together.

18. Method according to claim 17, in which the welding areas of the parts opposite each other are very near the edges of the layer of gel.
